(19) United States
(12) Patent Application Publication
(10) Pub. No.: US 2010/0210027 A9
(43) Pub. Date: Aug. 19, 2010

(54) METHOD FOR DETERMINING EFFECT OF PREFORMULATION FORMS ON THEIR DISSOLUTION RATES

(76) Inventors: Hongming Chen, Acton, MA (US); Hector Guzman, Jamaica Plain, MA (US); Colin R. Gardner, Concord, MA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/533,420

(22) PCT Filed: Nov. 4, 2003
(86) PCT No.: PCT/US03/35012

§ 371 Date: Apr. 29, 2005

Related U.S. Application Data
(60) Provisional application No. 60/423,365, filed on Nov. 4, 2002.
(60) Provisional application No. 60/423,366, filed on Nov. 4, 2002.

(51) Int. Cl. G01N 33/00

(52) U.S. Cl. 436/164

(57) ABSTRACT

Preformulation analysis and optimization comprising dissolution strudies.

### RELATED APPLICATIONS

This application claims the benefit of U.S. Provisional Application No. 60/423,365, filed Nov. 4, 2002, and U.S. Provisional Application No. 60/423,366, filed Nov. 4, 2002.

### FIELD OF INVENTION

This invention relates to methods of conducting preformulation studies. In one aspect, the invention relates to methods of rapidly determining the effect of various conditions on the solubility, dissolution, and stability of compounds.

### BACKGROUND OF THE INVENTION

Preformulation is a stage of formulation development during which the physical and/or chemical properties of a compound are studied in order to determine an optimum formulation. For example, the preformulation of an active pharmaceutical ingredient (API) typically involves the study of its solubility, stability, and other characteristics in order to determine how it can be formulated to provide a pharmaceutically acceptable dosage form with an economically acceptable shelf-life.

Preformulation is typically a tedious and costly process, as the properties of compounds can vary widely and are difficult to predict. The exceedingly high standards that govern the manufacture and sale of pharmaceuticals makes the preformulation of APIs even more difficult. Moreover, comprehensive preformulation studies of many APIs are not undertaken due to their high cost and limited availability. In addition, the drive to get pharmaceuticals to market as quickly as possible imposes time constraints that also prevent comprehensive preformulation studies.

The successful formulation of an API requires an understanding of its chemical and physical characteristics, and how they are affected by a variety of manufacturing, storage, and administration conditions. For example, if a compound is to be stored or used in a liquid formulation, its solubility properties must be determined in order to find, for example, liquids that can accommodate suitable concentrations. Solubility studies are also important in the development of liquid formulations from which compounds do not precipitate.

An understanding of the dissolution characteristics of compounds is similarly important. For example, if an API that is only bioavailable in solution is to be orally administered to patients in a solid dosage form, its ability to dissolve in the stomach and/or gastrointestinal tract must be determined.

The chemical and physical stability of a compound must also be known in order to formulate it effectively. For example, if an API is to be administered in solution (e.g., intravenously), its chemical stability in that solution must be understood. If the API is found to be more susceptible to decomposition when in solution, preformulation studies can identify that fact early, so that research efforts can focus on developing, for example, a solid form of the compound that can be readily dissolved in a pharmaceutically acceptable carrier immediately prior to administration. In another example, identification of the pH at which an API is most stable in solution can aid in the development of liquid formulations with long shelf lives.

Other useful information obtained by preformulation studies includes, but is not limited to, the effect of mechanical stress on compounds. For example, it is important to understand how the compression and shear forces to which an API may be subjected during manufacture affect it.

In sum, formulating a compound to provide an optimum balance of properties (e.g., solubility, dissolution, and stability) while minimizing the amount of the compound necessary to achieve its purpose can be a difficult task. A need therefore exists for rapid and systematic preformulation methods that require the use of only small amounts of compounds.

### SUMMARY OF THE INVENTION

This invention is directed, in part, to preformulation methods that can be used to determine the physical and/or chemical properties of a given compound (herein referred to as the “compound-of-interest”). For example, the invention provides a method of determining how the form (e.g., particle size, crystal form, and habit) of solid compounds-of-interest affect their solubility, dissolution, and chemical and physical stability.

The invention also provides a method of rapidly and systematically determining how characteristics of a compound-of-interest are affected by various conditions. Examples of characteristics include, but are not limited to, solubility, dissolution, hygroscopicity, and chemical and physical stability. Specific characteristics include, but are not limited to, dissolution profile, intrinsic dissolution, solution-state chemical stability (e.g., susceptibility to oxidation, hydrolysis, photolysis, and isomerization), solid-state chemical and physical stability (e.g., susceptibility to polymorphism), and wettability. Examples of conditions include, but are not limited to, pH, ionic strength, counter-ion concentration, relative humidity (e.g., moisture), radiation, oxidative conditions, mechanical stress, and temperature. Of course, the effect of the amount of time for which a compound-of-interest is exposed to one or more conditions can also be determined.

The invention further provides a method of identifying conditions that can enhance the bioavailability and/or the chemical or physical stability of a compound-of-interest.

The invention also encompasses a method of rapidly and systematically determining how characteristics of a compound-of-interest are affected when it is placed in contact with one or more chemical compounds such as, but not limited to, excipients and additives.

Specific methods of the invention utilize automated means of preparing large arrays of samples, each of which comprises a controlled amount of a compound-of-interest and an excipient. Automated means may also be used to expose specific samples in the arrays to varying conditions, and to rapidly analyze the samples to determine how those conditions affect a given chemical or physical property.

### BRIEF DESCRIPTION OF THE FIGURES

Aspects of certain embodiments of the invention may be understood from the figures included herein.

FIG. 1 provides a schematic of a specific method used to determine the effect of pH on the solubility of a compound-of-interest.

FIG. 2 provides an illustration of a template used for solubility and stability determination of the samples. Variation of pH in each sample is indicated by the horizontal labels, and variation of counter-ion concentration is indicated by the vertical labels.

FIG. 3 shows a data set obtained by the method outlined in FIG. 1.

FIG. 4 provides a schematic of another specific method used to determine the effect of pH on the solubility of a compound-of-interest, which utilizes turbidity measurements.

FIG. 5 shows a data set obtained by the method outlined in FIG. 4.

FIG. 6 provides a schematic of a specific method of the invention that can be used to determine the effect of pH on the dissolution profile of a compound-of-interest.

FIG. 7 shows a data set obtained by the method outlined in FIG. 6.

FIG. 8 shows a data set obtained by another method having the steps outlined in FIG. 6.

FIG. 9 provides a schematic of a specific method of the invention that can be used to determine the effect of pH on the stability of a compound-of-interest.

FIG. 10 provides a schematic of a specific method of the invention that can be used to evaluate the solid-state stability of a compound-of-interest.

### DEFINITIONS

As used herein and unless otherwise indicated, the term “array,” when used to refer to a plurality of objects (e.g., samples), means a plurality of objects that are organized physically or indexed in some manner (e.g., with a physical map or within the memory of a computer) that allows the ready tracking and identification of specific members of the plurality. Typical arrays of samples comprise at least 6, 12, 24, 94, 96, 380, 384, 1530, or 1536 samples.

As used herein and unless otherwise indicated, the term “excipient” refers to a compound that may potentially be combined with a compound-of-interest to provide a formulation of the compound-of-interest. Excipients can be liquid or solid. Examples of excipients include, but are not limited to diluents, binders, lubricants, stabilizing and neutralizing agents (e.g., antioxidants), and packaging and processing reagents.

As used herein and unless otherwise indicated, the term “condition” means the physical or chemical environment to which a compound-of-interest or sample is subjected. Examples of conditions include, but are not limited to, pH, ionic strength, counter-ion concentration, moisture (e.g., humidity), radiation (e.g., UV, visible, and IR light), oxidative conditions, mechanical stress (e.g., pressure and shear), temperature, and time.

As used herein and unless otherwise indicated, the term “controlled amount” refers to an amount of a compound that is weighed, aliquotted, or otherwise dispensed in a manner that attempts to control the amount of the compound. Preferably, a controlled amount of a compound differs from a predetermined amount by less than about 15, 14, 13, 12, 11, 10, 9, 8, 7, 6, 5, 4, 3, 2, or 1 percent of the predetermined amount. For example, if one were to dispense, handle, or otherwise use 100 micrograms of a compound-of-interest, a controlled amount of that compound-of-interest would preferably weight from about 85 micrograms to about 115 micrograms, from about 90 micrograms to about 110 micrograms, from about 95 micrograms to about 105 micrograms, or from about 99 micrograms to about 101 micrograms. Typically, the precise mass of the controlled amount is determined after being dispensed.

As used herein and unless otherwise indicated, the term “controlled relative humidity” refers to humidity that is maintained at a predetermined level. Preferably, a controlled relative humidity differs from a predetermined level by less than about 10, 9, 8, 7, 6, 5, 4, 3, 2, or 1 percent of the predetermined level.

As used herein and unless otherwise indicated, the term “form” encompasses the physical and chemical forms of a compound. Examples of physical forms include, but are not limited to, solid, liquid (e.g., oil), and gas. The physical form of a solid encompasses, but is not limited to, particle size (e.g., the average particle size or mean distribution of particle sizes of a powder), whether or not a compound is crystalline or amorphous or the degree to which it may be one or the other, the crystal form of a crystalline compound (i.e., its crystal structure), crystal habit, and color. Chemical forms of a compound include, but are not limited to, salts, free-bases, solvates (e.g., hydrate), co-crystals, and clathrates.

As used herein and unless otherwise indicated, the term “property” means a physical or chemical characteristic of a sample. Specific properties are those that relate to the efficacy, safety, stability, processing characteristics, or utility of compounds. Examples of properties include, but are not limited to, solubility, dissolution, intrinsic dissolution, chemical and physical stability, permeability, hygroscopicity, wettability, crystal form and habit, chirality, partitioning, compressibility, compactability, flow characteristics, color, taste, smell, absorption, bioavailability, toxicity, metabolic profile, and potency.

As used herein and unless otherwise indicated, the term “sample” refers to an isolated amount of a compound or composition. A typical sample comprises a controlled amount of a compound-of-interest and a solvent (e.g., pH buffer) and/or an excipient. Specific samples comprise a compound-of-interest in an amount less than about 25 mg, 1 mg, 500 micrograms, 250 micrograms, 100 micrograms, 50 micrograms, 25 micrograms, 10 micrograms, 5 micrograms, 1 microgram, or 0.5 micrograms. A sample can be contained in container (e.g., a jar, vial, or well), or can be deposited or adsorbed on a surface. The term “sample” includes replicates, e.g. n=2, 3, 4, 5, 6 or more.

As used herein and unless otherwise indicated, the term “stability” refers to the ability of a compound or composition to resist change (e.g., degradation, decomposition, isomerization, water content, or color change), when exposed to chemicals, light, heat, and mechanical stress. For example, the term “stability” encompasses the resistance of a compound or composition to the absorption of water. The term “stability” further encompasses the resistance of a solid compound or composition to a change in form, such as, but not limited to, a phase change, habit change, or polymorphic transition.

### DETAILED DESCRIPTION OF THE INVENTION

Preformulation studies represent an important step in the life-cycle management of any useful composition. Following the discovery and early-lead optimization of a compound-of-interest, preformulation studies provide vital information and direction for subsequent development of a suitable formulation and preparation for pre-clinical studies. Whereas the early-lead optimization attempts to identify an optimum compound or compounds for further development as a compound-of-interest, the preformulation methods described herein can provide information regarding solubility, dissolution, stability, hygroscopicity, as well as many other important considerations during formulation design of a compound-of-interest. The information gathered through the methods of preformulation analysis allow formulation development to proceed more efficiently, and more successfully than without such methods. In fact, the applicants have found surprising success in developing formulations for compounds-of-interest by utilizing the preformulation methods described herein. Specific combinations of excipients are identified quickly as useful formulations which can be used to improve bioavailability, dissolution and other important aspects of pharmaceuticals, nutraceuticals, alternative medicaments, and the like.

This invention is based, in part, on a discovery that systematic and parallel methods can be used to rapidly and efficiently conduct preformulation studies of a wide variety of compounds-of-interest including, but not limited to, APIs.

A first embodiment of the invention encompasses a method of determining how the solubility of a solid compound-of-interest is affected by its form, which comprises: (a) preparing an array of samples, each comprising a controlled amount of the compound-of-interest, wherein the form of the compound-of-interest in at least two of the samples is different; (b) forming a liquid portion of each sample by adding a solvent to each sample; and (c) determining how much compound-of-interest dissolved in the liquid portion of each sample.

Another embodiment of the invention encompasses a method of determining how the dissolution of a solid compound-of-interest is affected by its form, which comprises: (a) preparing an array of samples, each comprising a controlled amount of the compound-of-interest, wherein the form of the compound-of-interest in at least two of the samples is different; (b) forming a liquid portion of each sample by adding a solvent to each sample; and (c) determining how much compound-of-interest dissolved in the liquid portion of each sample as a function of time.

A specific method of this embodiment comprises: (a) preparing a first sub-array of samples, each comprising a controlled amount of the compound-of-interest in a first form; (b) preparing a second sub-array of samples, each comprising a controlled amount of the compound-of-interest in a second form that differs from the first form; (c) forming a liquid portion of each sample in the first sub-array by adding a controlled amount of a solvent to each sample in the first sub-array at a time point that is unique to each sample in the first sub-array; (d) forming a liquid portion of each sample in the second sub-array by adding a controlled amount of a solvent to each sample in the second sub-array at a time point that is unique to each sample in the second sub-array but is the same as the time point at which solvent was added to a sample in the first sub-array; (e) separating the liquid portion of each sample in the first and second sub-arrays from any solid portion each sample may contain at a time point that is the same for each sample in the first and second sub-arrays; and (f) determining how much compound-of-interest dissolved in the liquid portion of each sample.

Another embodiment of the invention encompasses a method of determining how the stability of a solid compound-of-interest is affected by its form, which comprises: (a) preparing an array of samples, each comprising a controlled amount of the compound-of-interest, wherein the form of the compound-of-interest in at least two of the samples is different; (b) exposing the compound-of-interest in each sample to a condition that may affect the stability of the compound-of-interest; and (c) determining whether the form or chemical composition of the compound-of-interest in each sample changed.

Another embodiment of the invention encompasses a method of determining how the hygroscopicity of a solid compound-of-interest is affected by its form, which comprises: (a) preparing an array of samples, each comprising a controlled amount of the compound-of-interest, wherein the form of the compound-of-interest in at least two of the samples is different; (b) exposing the compound-of-interest in each sample to a controlled relative humidity for a period of time; and (c) determining the change in water content of the compound-of-interest in each sample.

Another embodiment of the invention encompasses a method of determining the effect of a condition on the solubility of a compound-of-interest, which comprises: (a) preparing an array of samples having a liquid portion, each comprising a controlled amount of the compound-of-interest and a solvent; (b) exposing each sample to a condition that differs for at least two samples in the array; and (c) determining how much compound-of-interest dissolved in the liquid portion of each sample.

Another embodiment of the invention encompasses a method of determining the effect of a condition on the dissolution of a compound-of-interest, which comprises: (a) preparing an array of samples having a liquid portion, each comprising a controlled amount of the compound-of-interest and a solvent; (b) exposing each sample to a condition that differs for at least two samples in the array; and (c) determining how much compound-of-interest dissolved in the liquid portion of each sample as a function of time.

A specific method of this embodiment comprises: (a) preparing a first sub-array of samples, each comprising a controlled amount of the compound-of-interest; (b) preparing a second sub-array of samples, each comprising a controlled amount of the compound-of-interest; (c) forming a liquid portion of each sample in the first sub-array by adding a solvent to each sample in the first sub-array at a time point that is unique to each sample in the first sub-array; (d) exposing each sample in the first sub-array to a first condition; (e) forming a liquid portion of each sample in the second sub-array by adding a solvent to each sample in the second sub-array at a time point that is unique to each sample in the second sub-array but is the same as the time point at which solvent was added to a sample in the first sub-array; (f) exposing each sample in the second sub-array to a second condition that differs from the first condition; (g) separating the liquid portion of each sample in the first and second sub-arrays from any solid portion each sample may contain at a time point that is the same for each sample in the first and second sub-arrays; and (h) determining how much compound-of-interest dissolved in the liquid portion of each sample.

Another embodiment of the invention encompasses a method of determining the effect of a condition on the stability of a compound-of-interest, which comprises: (a) preparing an array of samples, each comprising a controlled amount of the compound-of-interest; (b) exposing each sample to a condition that differs for at least two samples in the array; and (c) determining whether the form or chemical composition of the compound-of-interest in each sample changed.

Another embodiment of the invention encompasses a method of determining the effect of a condition on the hygroscopicity of a compound-of-interest, which comprises: (a) preparing an array of samples, each comprising a controlled amount of the compound-of-interest; (b).exposing the compound-of-interest in each sample to a controlled relative humidity for a period of time and to an additional condition that differs for at least two samples in the array; and (c) determining the change in water content of the compound-of-interest in each sample.

Another embodiment of the invention encompasses a method of determining the effect of an excipient on the solubility of a compound-of-interest, which comprises: (a) preparing an array of samples having a liquid portion, each comprising a controlled amount of the compound-of-interest, a solvent, and an excipient, wherein the excipient or the amount of excipient differs for at least two of the samples; and (b) determining how much compound-of-interest dissolved in the liquid portion of each sample.

Another embodiment of the invention encompasses a method of determining the effect of an excipient on the dissolution of a compound-of-interest, which comprises: (a) preparing an array of samples having a liquid portion, each comprising a controlled amount of the compound-of-interest, a solvent, and an excipient, wherein the excipient or the amount of excipient differs for at least two of the samples; and (b) determining how much compound-of-interest dissolved in the liquid portion of each sample as a function of time.

A specific method of this embodiment comprises: (a) preparing a first sub-array of samples, each comprising a controlled amount of the compound-of-interest and a first excipient; (b) preparing a second sub-array of samples, each comprising a controlled amount of the compound-of-interest and a second excipient that differs from the first excipient and/or is provided in a different amount than the first excipient; (c) forming a liquid portion of each sample in the first sub-array by adding a solvent to each sample in the first sub-array at a time point that is unique to each sample in the first sub-array; (d) forming a liquid portion of each sample in the second sub-array by adding a solvent to each sample in the second sub-array at a time point that is unique to each sample in the second sub-array but is the same as the time point at which solvent was added to a sample in the first sub-array; (e) separating the liquid portion of each sample in the first and second sub-arrays from any solid portion each sample may contain at a time point that is the same for each sample in the first and second sub-arrays; and (f) determining how much compound-of-interest dissolved in the liquid portion of each sample.

Another embodiment of the invention encompasses a method of determining the effect of an excipient on the stability of a compound-of-interest, which comprises: (a) preparing an array of samples, each of which comprises a controlled amount of the compound-of-interest and an excipient, wherein the excipient or the amount of excipient differs for at least two of the samples; (b) exposing the samples to a condition that may affect the stability of the compound-of-interest; and (c) determining whether the form or chemical composition of the compound-of-interest in each sample changed.

Another embodiment of the invention encompasses a method of determining the effect of an excipient on the hygroscopicity of a compound-of-interest, which comprises: (a) preparing an array of samples, each of which comprises a controlled amount of the compound-of-interest and an excipient, wherein the excipient or the amount of excipient differs for at least two of the samples; (b) exposing the samples to a controlled relative humidity for a period of time; and (c) determining the change in water content of the compound-of-interest in each sample.

Another embodiment of the invention encompasses a method of determining the effect of a controlled relative humidity on the form of a compound-of-interest, which comprises: (a) preparing an array of samples, each of which comprises a controlled amount of the compound-of-interest, wherein the form of the compound-of-interest in at least two of the samples is different; (b) exposing the compound-of-interest in each sample to a controlled relative humidity for a period of time; and (c) determining the change in form, if any, of the compound-of-interest in each sample.

Another embodiment of the invention emcompasses a method of determining the existence of a synergistic effect of mixtures of excipients on the solubility of a compound-of-interest, which comprises: (a) preparing an array of samples, each of which comprises a controlled amount of the compound-of-interest and at least two excipients; (b) determining the solubility of the compound-of interest in each mixture of two or more excipients; and (c) comparing the solubility of the compound-of-interest in each mixture with the solubility of the compound-of-interest in each single excipient of the mixture.

This embodiment provides a method to determine which mixtures of excipients yield unexpectedly high or low solubility. For example, if the solubility of a compound-of-interest is 5 mg/mL in excipient A, the solubility of the compound-of-interest is 1 mg/mL in excipient B, and the solubility of the compound-of-interest in a mixture of excipients A and B is 15 mg/mL, the discovery of the mixture comprising A and B can be useful in the design of a formulation for the compound-of interest. Similarly, a synergistic effect which decreases the solubility can also be of particular importance. The ability to change the solubility of a compound-of-interest simply by changing the excipient mixture used is a powerful tool in the quest for improved formulations. Synergistic effects of other characteristics such as hygroscopicity, dissolution, and stability can also be found using methods of the present invention.

Each of the various embodiments of the invention preferably utilizes microarray technology used in one or more of the methods and systems referred to as FAST®, CRYSTALMAX™, and SFinX™. The methods and systems referred to as FAST® are described in U.S. patent application Ser. No. 09/628,667, filed Jul. 28, 2000, the entirety of which is incorporated herein by reference. The methods and systems referred to as CRYSTALMAX™ are described in U.S. patent application Ser. No. 09/756,092, filed Jan. 8, 2001, and International Publication WO01/51919, published on Jul. 19, 2001, both of which are incorporated herein in their entireties by reference. The methods and systems referred to as SFinX™ are disclosed in U.S. Provisional Application No. 60/423,366, the entirety of which is incorporated herein by reference.

In particular methods of the invention, arrays of samples containing a controlled amount of a compound-of-interest and a solvent and/or excipient are prepared and screened. The results obtained from one array can be used to refine or focus additional experiments aimed at uncovering information useful in the formulation of the compound-of-interest.

Compounds-of-Interest

Embodiments of this invention can be used for the discovery of useful and novel formulations of a wide variety of compounds-of-interest. Examples of compounds-of-interest include, but are not limited to, active components of pharmaceuticals, inactive components of pharmaceuticals (e.g., excipients), dietary supplements, alternative medicines, nutraceuticals, sensory compounds, agrochemicals, consumer formulations, and industrial formulations. Particular compounds-of-interest are active pharmaceutical ingredients (APIs). Compounds-of-interest may be solid or liquid at room temperature.

Pharmaceuticals are substances that have a therapeutic, disease preventive, diagnostic, or prophylactic effect when administered to an animal or a human, and include prescription and over-the-counter drugs. Some examples of compound-of-interests are listed in 2000 Med Ad News 19:56-60 and The Physicians Desk Reference, 56^{th} ed. (2002). Examples of veterinary pharmaceuticals include, but are not limited to, vaccines, antibiotics, growth enhancing excipients, and dewormers. Other examples of veterinary pharmaceuticals are listed in The Merck Veterinary Manual, 8^{th} ed., Merck and Co., Inc., Rahway, N.J., 1998; The Encyclopedia of Chemical Technology, 24 Kirk-Othomer (4^{th} ed. at 826); and A. L. Shore and R. J. Magee, Veterinary Drugs in ECT, vol. 21 (2^{nd} ed.) (American Cyanamid Co.).

Dietary supplements are non-caloric or insignificant-caloric substances administered to an animal or a human to provide a nutritional benefit or non-caloric or insignificant-caloric substances administered in a food to impart the food with an aesthetic, textural, stabilizing, or nutritional benefit. Dietary supplements include, but are not limited to, fat binders, such as caducean; fish oils; plant extracts, such as garlic and pepper extracts; vitamins and minerals; food additives, such as preservatives, acidulents, anticaking excipients, antifoaming excipients, antioxidants, bulking excipients, coloring excipients, curing excipients, dietary fibers, emulsifiers, enzymes, firming excipients, humectants, leavening excipients, lubricants, non-nutritive sweeteners, food-grade solvents, thickeners; fat substitutes, and flavor enhancers; and dietary aids, such as appetite suppressants. Examples of dietary supplements are listed in (1994) The Encyclopedia of Chemical Technology, 11 Kirk-Othomer (4^{th} ed. at 805-833). Examples of vitamins are listed in (1998) The Encyclopedia of Chemical Technology, 25 Kirk-Othomer (4^{th} ed. at 1) and Goodman & Gilman's: The Pharmacological Basis of Therapeutics, 9^{th} ed., Joel G. Harman and Lee E. Limbird, eds., McGraw-Hill, 1996 p.1547. Examples of minerals are listed in The Encyclopedia of Chemical Technology, 16 Kirk-Othomer (4^{th} ed. at 746) and “Mineral Nutrients” in ECT 3^{rd} ed., vol 15, pp. 570-603, by C. L. Rollinson and M. G. Enig, University of Maryland.

Alternative medicines are substances, preferably natural substances, such as herbs or herb extracts or concentrates, administered to a subject or a patient for the treatment of disease or for general health or well being, which do not require approval by the FDA. Examples of alternative medicines include, but are not limited to, ginkgo biloba, ginseng root, valerian root, oak bark, kava kava, echinacea, harpagophyti radix. Other examples are listed in The Complete German Commission E Monographs: Therapeutic Guide to Herbal Medicine, Mark Blumenthal et al. eds., Integrative Medicine Communications 1998.

Nutraceuticals are foods or food products having both caloric value and pharmaceutical or therapeutic properties. Example of nutraceuticals include, but are not limited to, garlic, pepper, brans and fibers, and health drinks. Other examples are listed in M. C. Linder, ed. Nutritional Biochemistry and Metabolism with Clinical Applications, Elsevier, N.Y., 1985; Pszczola et al., 1998 Food Technology 52:30-37 and Shukla et al., 1992 Cereal Foods World 37:665-666.

Excipients

Certain embodiments of the invention are directed to the high-throughput determination of the effects excipients have on various properties of compounds-of-interest and compositions comprising them. Examples of excipients include, but are not limited to diluents, binders, lubricants, stabilizing and neutralizing agents (e.g., antioxidants), and packaging and processing reagents.

Excipients that can be used in methods of the invention may be novel, but commercially available excipients that are generally recognized as safe (GRAS) are preferred. Examples of excipients that can be used in various embodiments of the invention include, but are not limited to: acidulents, such as lactic acid, hydrochloric acid, and tartaric acid; solubilizing excipients, such as non-ionic, cationic, and anionc surfactants; absorbents, such as bentonite, cellulose, and kaolin; alkalizing excipients, such as diethanolamine, potassium citrate, and sodium bicarbonate; anticaking excipients, such as calcium phosphate tribasic, magnesium trisilicate, and talc; antimicrobial excipients, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and zein; buffering excipients, such as sodium phosphate, malic acid, and potassium citrate; chelating excipients, such as EDTA, malic acid, and maltol; coating excipients, such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as collodial silicon dioxide, croscarmellose sodium, magnesium aluminum silicate, potassium polacrilin, and sodium starch glycolate; dispersing excipients, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying excipients, such as anionic emulsifying wax, monoethanolamine, and medium chain triglycerides; flavoring excipients, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitosterate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing excipients, such as cyclodextrins, albumin, xanthan gum; and tonicity excipients, such as glycerol, dextrose, potassium chloride, and sodium chloride; and mixtures thereof. Other excipients, such as binders and fillers, are known to those of ordinary skill in the art. See, e.g., Remington's Pharmaceutical Sciences, 18^{th} ed., ed. Alfonso Gennaro, Mack Publishing Co. Easton, Pa., 1995; Handbook of Pharmaceutical Excipients, 3^{rd} Edition, ed. Arthur H. Kibbe, American Pharmaceutical Association, Washington D.C. 2000.

Formulations can comprise one or more excipients. For example, a formulation can comprise one, two, three, four, five, or more excipients, in addition to a compound-of-interest. A binary formulation comprises two excipients and a compound-of interest, a ternary formulation comprises three excipients and a compound-of-interest, and so on.

Additional non-limiting examples of excipients include: acetylated monoglycerides, C8/C10 diesters of propylene glycol of coconut oil, caprylic/capric triglyceride, castor oil, coconut oil, corn oil, cottonseed oil, diacetylated monoglycerides, ethylene glycol, gelucire 33/01, glycerin, glyceryl linoleate, glyceryl oleate, glyceryl ricinoleate, hydrogenated coconut oil, linoleic acid, mineral oil, mono-/diglyceride from coconut oil (C8/C10), monoolein:propylene glycol (90:10), myristyl alcohol, oleic acid, olive oil, palm oil, peanut oil, PEG 60 almond glycerides, PEG-6 isostearate, PEG-8 caprylic/capric glyceride, caprylocaproyl macrogol-8 glycerides, poloxamer 331, PEG 1000, PEG 200, PEG 300, PEG 400, PEG 600, polyglycerol-3-diisostearate, polyglycerol-6 dioleate, polyoxyethylene glycerol trioleate, polyoxyl 30 castor oil, polyoxyl 35 castor oil, polyoxyl 40 castor oil, polyoxyl 40 stearate, polypropylene glycol 2000, polypropylene glycol 725, polysorbate 20, polysorbate 40, polysorbate 60, polysorbate 80, propylene glycol, propylene glycol monocaprylate, propylene glycol monolaurate, safflower oil, sesame oil, sorbitan monolaurate, sorbitan monooleate, sorbitan trioleate, soybean oil, sunflower seed oil, triacetin, triethanolamine, trilaurin, water, benzyl alcohol, benzyl benzoate, diethylene glycol monoethyl ether, ethylene glycol monoethyl ether, isopropanolamine, cetearyl alcohol, cetyl alcohol, cetyl esters wax, acetic acid, ethanol, cyclodextrin, poloxamer 188, and sodium hydroxide.

Array Preparation and Analysis

According to this invention, the high-throughput preformulation study of a compound-of-interest is achieved using arrays of samples, each of which contains a controlled amount of the compound-of-interest. The samples may contain additional compounds (e.g., excipients), depending on the particular embodiment of the invention.

For example, one embodiment of the invention allows the rapid and efficient determination of the effect of the form (e.g., its crystalline structure) of a compound-of-interest on its properties. A particular method of this embodiment provides information regarding the effect of the compound-of-interest's crystalline form on its solubility in one or more solvents by using an array of samples comprising a controlled amount of the compound-of-interest in its various forms and a controlled amount of the solvent(s) under investigation.

In another embodiment, the effect of a condition on the chemical composition, physical form, or properties (e.g., solubility, dissolution, hygroscopicity, and stability) of a compound-of-interest is determined. Examples of conditions include, but are not limited to, radiation (e.g., light), heat, or mechanical force. In methods of this embodiment, each sample in the array may simply contain a controlled amount of compound-of-interest, and may differ only with regard to the condition(s) to which they are exposed.

In another embodiment of the invention, the effect of solvents, ionic conditions (e.g., counter-ion concentration and ionic strength), or acidic conditions (e.g., pH) on a compound-of-interest are determined. In that case, samples in an array will comprise a controlled amount of a solvent, an ionic solution (e.g., an aqueous ionic solution), or a pH buffer.

In other embodiments of the invention, the effect of one or more excipients or amounts of excipients on the properties of a compound-of-interest are determined. In those embodiments, each sample in an array will comprise a controlled amount of the compound-of-interest and a controlled amount of an excipient. If the effect of the excipient on the solubility of the compound-of-interest is under investigation, each sample will further comprise a controlled amount of the solvent.

As made clear in the more detailed discussions of the various embodiments of the invention provided below, most methods of the invention utilize an array of samples that differ from each other by the concentration of the compound-of-interest and/or the solvent(s) or excipient(s) they contain. In certain circumstances, each sample within an array will differ from the others. In other circumstances, it may be desirable to provide redundancy within the array to reduce experimental error or to allow the testing and/or preparation of identical samples at different times (e.g., to determine dissolution profiles, as discussed below).

The samples of specific arrays comprise less than 25 mg, 1 mg, 500 micrograms, 250 micrograms, 100 micrograms, 50 micrograms, 25 micrograms, 10 micrograms, 5 micrograms, 1 microgram, or 0.5 micrograms of the compound-of-interest or solid excipient(s). The samples of specific arrays may comprise less than about 5 ml, 2.5 ml, 1 ml, 500 microliters, 250 microliters, 100 microliters, 50 microliters, 25 microliters, 10 microliters, 5 microliters, 1 microliter, or 0.5 microliters of a solvent or liquid excipient(s). In typical embodiments of the invention, containers, such as tubes, vials, or wells, are used to hold the samples. In addition, one or more of the containers are typically provided as controls to aid in the automated screening of arrays. Typical controls will contain no sample, will contain only compound-of-interest, or will contain only solvent or excipient.

If samples in an array comprise both solids and liquids, the samples are typically prepared by adding a controlled amount of one or more of the solids to each container (except, perhaps, one or more used as controls), after which controlled amounts of the liquid(s) are added to each container. Of course, the particular order by which samples are prepared can be varied as desired. For example, one or more liquid excipients can be added before the controlled amount of one or more solids is added, or the controlled amount of one or more solids can be dispensed before contacting with one or more liquid excipients. Stir bars or tumblers can optionally be added to each container to ensure that the sample mixtures are well mixed. Samples may also be heated to help the compound-of-interest in each sample dissolve to the extent possible. In some embodiments of the invention, the formation of arrays is done using automated techniques that allow the rapid preparation of large arrays.

If the compound-of-interest is a liquid at room temperature, it can be transferred to the tubes, wells, or other containers forming an array using conventional techniques (e.g., using a pipettor). If it is a viscous liquid, it can be transferred using a positive displacement pump. The controlled transfer of a viscous liquid may be facilitated by heating the pump and transfer lines while in the pump and/or while being transferred. Solid compounds-of-interest can be transferred using conventional methods, although preferred techniques are disclosed in U.S. Provisional Application Nos. 60/423,377, 60/424,001, and 60/430,089 filed on Nov. 4, 2002, Nov. 6, 2002, and Dec. 2, 2002, respectively, the entireties of which are incorporated herein by reference. Solvents and excipients can be transferred to the tubes, wells, or other containers forming an array using the same methods and devices used to transfer the compound-of-interest.

Solubility Analysis

Methods of the invention can be used to rapidly and systematically determine the solubility of a compound-of-interest in a variety of different solvents such as, but not limited to, aqueous solvents (e.g., pH buffers and solutions comprising concentrations of certain ions and/or excipients) and organic solvents (e.g., alcohols, such as methanol and ethanol; alkanes, such as hexanes; and aromatics, such as benzene, toluene, THF, and pyridine; and others, such as DMSO). Methods of the invention can also be used to determine how a variety of excipients that may be used in the manufacture, storage, or final formulation of a compound-of-interest affect its solubility. Additional methods of the invention can be used to determine how the effect of certain conditions (e.g., temperature) affect the solubility of a compound-of-interest or composition comprising a compound-of-interest.

In each of these cases, an array of samples comprising the compound-of-interest, an amount of solvent, and one or more optional excipients is prepared. Preferably, each sample is stirred and allowed to equilibrate before analyzed. If one exists; the solid portion as well as the liquid portion of a sample can be analyzed. For samples of very small volume (e.g., about 20 microliters or less), nephelometry is preferably used to determine solubility. In such circumstances, small amounts of solvents in which a compound-of-interest is highly soluble, such as, but not limited to, dimethyl sulfoxide (DMSO) can be used to aid in the transfer of controlled amounts of compound-of-interest. If the volume of a sample is large enough to separate its solid and liquid portions by filtration or centrifuge, standard techniques such as, but not limited to, high performance liquid chromatography (HPLC), nuclear magnetic resonance spectroscopy (NMR) (e.g., ¹H and ¹³C NMR), Raman spectroscopy (e.g., resonance Raman spectroscopy), and absorption and emission spectroscopy (e.g., infrared, visible,ultraviolet absorption and emission, and fluorescence) can be used to determine how much solid dissolved.

The effect of conditions such as pH, ionic strength, and counter-ion concentration on the solubility of a compound-of-interest can be determined by selecting the appropriate solvent. Examples of pH buffers that can be used in methods of the invention have pHs of from about 7.5 to about 1.0 , from about 7.0 to about 5.0 , and from about 6.8 to about 5.5 . Other examples have pHs of from about 6.5 to about 10.0 , from about 7.0 to about 9.0 , and from about 7.5 to about 8.5 . It should also be noted that the dissolution of the compound-of-interest itself may affect the pH of its environment in a way that affects the ultimate solubility and other properties of the compound-of-interest. This, too, can be studied using methods of the invention. Furthermore, varying concentrations of inorganic ions such as, but not limited to, Li⁺, Na⁺, K⁺, Mg²⁺, Ca²⁺, F⁻, Cl⁻, Br⁻, and I⁻, and organic ions, such as, but not limited to, tartrate, phosphate, malate, succinate, ascorbate, besylate, adipate, and fumarate, can be added to solutions (e.g., aqueous solutions) in order to determine their effect on the solubility of the compound-of-interest. Also, various excipients with intrinsic buffer capacities can be studied to determine their effect on the solubility of the compound-of-interest. The effect of other conditions such as, but not limited to, temperature, radiation, relative humidity (e.g., moisture), and chemical (e.g., oxidative or reductive) conditions on the solubility of the compound-of-interest can be determined by exposing individual samples or groups of samples (e.g., sub-arrays) within an array to those conditions using techniques known in the art. For example, fiber optics can be used to expose individual samples to light, while heated blocks into which vials fit can be used to heat arrays of samples.

Dissolution Analysis

Methods of the invention can be used to rapidly and systematically determine the dissolution characteristics of a compound-of-interest in a variety of different solvents such as, but not limited to, aqueous solvents (e.g., pH buffers and solutions comprising concentrations of certain ions), excipients, and organic solvents (e.g., alcohols, such as methanol and ethanol; alkanes, such as hexanes; and aromatics, such as benzene, toluene, THF, and pyridine; and others, such as DMSO). Methods of the invention can also be used to determine how a variety of excipients that may be used in the manufacture, storage, or final formulation of a compound-of-interest affect its dissolution. Additional methods of the invention can be used to determine how the effect of certain conditions (e.g., temperature) affect the dissolution of a compound-of-interest or composition comprising a compound-of-interest.

In general, the dissolution of a solid compound-of-interest in a particular solvent (e.g., a pH buffer used to emulate gastric fluids) is determined by preparing an array of samples containing a controlled amount of the compound-of-interest and a controlled amount of the solvent, and measuring the concentration of the dissolved compound-of-interest as a function of time. The compound-of-interest's solubilized concentration as a function of time is referred to as its “dissolution profile,” and is distinguished from its “equilibrium solubility,” which refers to its solubilized concentration at equilibrium. It is also distinguished herein from the intrinsic dissolution of the compound-of-interest, which may also be determined using methods of this invention. The intrinsic dissolution of a compound-of-interest is the rate of change of solubilized compound as a function of time and surface area. It is typically measured under sink conditions in which total surface area does not change.

By using arrays of samples, methods of this invention allow the rapid determination of how a variety of different factors affect the dissolution profile and equilibrium solubility of a compound-of-interest or a composition comprising it. For example, methods of the invention can be used to determine the dissolution profile of a compound-of-interest in a variety of solvents by using arrays of samples that contain a controlled amount of compound-of-interest and solvent, but which vary with regard to the solvents they contain. Similarly, methods of the invention can be used to rapidly determine the effect excipients have on the dissolution profile of formulations comprising them by using arrays of samples containing a controlled amount of compound-of-interest and solvent, but which vary with regard to excipient. In these methods, controlled amounts of formulations made prior to the experiment, which comprise the compound-of-interest and at least one excipient, can be dispensed into sample containers (e.g., wells or vials) before the solvent is added to each.

In many instances, the dissolution of a compound-of-interest or composition can be determined by measuring, as a function of time, the concentration of the compound-of-interest in the solvent being used. For example, the dissolution of a compressed tablet encompasses the disintegration of the tablet, which can yield compound-of-interest in a powder (e.g., containing amorphous or crystalline particles of the compound-of-interest) that need not be soluble in the particular solvent used. The formation of powders, emulsions, and the like can be detected as a function of time using various methods, such as, but not limited to, computerized imaging and light scattering (e.g., nephelometry). If the dissolution profile of a compound-of-interest or composition comprising a compound-of-interest is to be determined by measuring how much of the compound-of-interest dissolves in the solvent as a function of time, methods such as, but not limited to HPLC, NMR (e.g., ¹H and ¹³C NMR), Raman spectroscopy (e.g., resonance Raman spectroscopy), X-ray spectroscopy, powder X-ray diffraction, absorption and emission spectroscopy (e.g., infrared, visible, and ultraviolet absorption and emission), differential scanning calorimetry (DSC), and thermogravimetric analysis (TGA) can be used.

Regardless of the analytical technique used, methods of determining the dissolution profile of a compound-of-interest or composition comprising a compound-of-interest require the collection of data at multiple times after the compound-of-interest or composition has been contacted with the solvent. This is readily done using automated computerized imaging, nephelometry, and light scattering techniques. However, if an accurate determination of how much compound-of-interest is dissolved in a solvent at a specific time is desired, it is preferred that the dissolution process be stopped at that particular time by, for example, separating the liquid and solid portions of a sample, after which the portions can be analyzed separately. Typically, the liquid portion is analyzed, although the analysis of the solid portion by Raman spectroscopy, X-ray powder diffraction, or other methods can also provide useful preformulation information. There are also spectroscopic techniques, however, which allow for the in situ measurement of dissolution without separation of the liquid and solid portions. In another embodiment of the present invention, the dissolution of a compound-of-interest can be determined without the separation of the liquid and solid portions.

In another method of the invention, the time for which a compound-of-interest or composition is exposed to a solvent is controlled by preparing an array of samples, each of which comprises the compound-of-interest and any optional excipients. A controlled amount of the solvent is then added to samples in the array at different times, after which the dissolution process is stopped for all of them at the same time. A controlled amount of the liquid excipient is added to each of the samples in the new array, but at different times. For example, at time 0, solvent is added to sample number 1; at time 1, solvent is added to sample number 2; and so on. At a final time, the solid—if any—and liquid portions of each sample in the new array are separated (e.g., by filtration or centrifuge), and the amount of compound-of-interest in the liquid portion of each is determined and correlated with time. This can be done simultaneously for multiple solvents or excipients by using sub-arrays within an array, each of which contains multiple samples comprising the same compounds in the same amounts. The sub-arrays will differ with regard to the variable(s) being studied in connection with the dissolution profile., e.g., solvent, condition, or excipient.

Stability Analysis

Methods of the invention can be used to rapidly and systematically determine the physical and chemical stability characteristics of liquid and solid compounds-of-interest under a variety of conditions. For example, the stability of a compound-of-interest dissolved or suspended in different solvents such as, but not limited to, aqueous solvents (e.g., pH buffers and solutions comprising concentrations of certain ions) and organic solvents (e.g., alcohols, such as methanol and ethanol; alkanes, such as hexanes; and aromatics, such as benzene, toluene, THF, and pyridine; and others, such as DMSO) can be determined. Methods of the invention can also be used to determine how excipients that may be used in the manufacture, storage, or final formulation of a compound-of-interest affect its physical and chemical stability. For example, the effect of various excipients or amounts of excipients on the solid state stability of a compound-of-interest may be determined. Additional methods of the invention can be used to determine how the effect of certain conditions (e.g., temperature) affect the physical and chemical stability of a compound-of-interest or composition comprising a compound-of-interest.

As discussed elsewhere herein, the term “stability” is used to refer to a compound-of-interest's resistance to change. For example, the physical stability of a compound-of-interest having a particular solid form refers to the compound-of-interest's ability to retain that form over time and/or when placed in contact with various chemical and/or environmental conditions. Similarly, the chemical stability of a compound-of-interest reflects its ability to resist chemical degradation (e.g., reaction, decomposition, or isomerization) over time and/or when placed in contact with various chemical and/or environmental conditions. Thus, embodiments of the invention that are directed to determining the stability of a compound-of-interest include, but are not limited to, methods of determining the compatibility of the compound-of-interest with various excipients (liquid and solid) and solvents.

In general, the physical stability of a particular form of a compound-of-interest in a particular solvent (e.g., a pH buffer used to emulate gastric fluids) or when exposed to a particular condition (e.g., light or heat) is determined by first characterizing that form using a method such as, but not limited to, Raman spectroscopy, powder X-ray diffraction, differential scanning calorimetry (DSC), optical microscopy, or birefringence screening. An array of samples containing a controlled amount of the compound-of-interest is then prepared and contacted with the solvent or condition, and, after varying amounts of time, the solvent or condition is removed and the samples (or the removed solvent portions) are analyzed. In another method, the solvent and/or condition used to affect the stability of the compound-of-interest is applied to samples within an array at various times, and then removed from all of the samples at the same time, thereby providing an array of compounds-of-interest, each of which was exposed to the solvent and/or condition for a different amount of time. The compounds-of-interest are subsequently analyzed (e.g., by Raman spectroscopy, X-ray powder diffraction, differential scanning calorimetry (DSC), or optical microscopy) to determine if their form changed.

The chemical stability of a compound-of-interest is determined in much the same way. In this case, methods such as, but not limited to, HPLC, NMR (e.g., ¹H and ¹³C NMR), Raman spectroscopy (e.g., resonance Raman spectroscopy), X-ray spectroscopy, powder X-ray diffraction, absorption and emission spectroscopy (e.g., infrared, visible, and ultraviolet absorption and emission), birefringence screening, differential scanning calorimetry (DSC), gravimetric and thermogravimetric analysis (TGA) can be used to determine whether the compound-of-interest in a particular sample was chemically altered by the solvent, excipient, or condition to which it was exposed. The preparation and analysis of arrays of such samples, each of which can differ with regard to solvent, excipient, condition, or time of exposure, is used to rapidly and efficiently obtain preformulation information.

Hygroscopicity Analysis

In many cases, the ability or inability of a compound-of-interest to absorb water from its environment must be understood to provide a suitable formulation. For example, the absorption of moisture by a compound-of-interest may facilitate the chemical degradation of the compound-of-interest, may induce polymorphism of the compound-of-interest, or may affect the physical or chemical properties of a composition comprising the compound-of-interest (e.g., the absorption of moisture may cause a tablet comprising the compound-of-interest to disintegrate while on the shelf).

Advantageously, this invention encompasses methods of rapidly and systematically determining how the physical form of a compound-of-interest affects it hygroscopicity or wettability. Other methods of the invention can be used to determine how various manufacturing and storage conditions affect the hygroscopicity of a compound-of-interest. Still others can be used to determine the effect of excipients and amounts of excipients on the hygroscopicity of a compound-of-interest.

In a particular embodiment of the invention, samples in an array are exposed to a first controlled relative humidity for a specific amount of time (e.g., sufficient for the samples to equilibrate), after which they are analyzed to determine how much, if any, water was absorbed. The samples are then exposed to a second controlled relative humidity, which is greater than the first, for a specific amount of time (e.g., sufficient for the samples to equilibrate). After equilibration, the samples are analyzed again. This process can be repeated using any number of different relative humidity levels to provide a hygroscopicity profile for each of the samples. Using this information, the effect of the form of a compound-of-interest, conditions (e.g., temperature or radiation), excipients, and the like (all of which can differ among samples in an array) on the hygroscopicity of compound-of-interest can be determined.

The absorption of water by a compound-of-interest or a composition comprising a compound-of-interest can be determined by a variety of techniques known to those of ordinary skill in the art. Examples of such techniques include, but are not limited to, NMR (e.g., ¹H and ¹³C NMR), X-ray spectroscopy, powder X-ray diffraction, absorption and emission spectroscopy (e.g., infrared absorption and emission), differential scanning calorimetry (DSC), and thermogravimetric analysis (TGA).

### EXEMPLIFICATION

Certain embodiments of this invention, as well as certain novel advantages of this invention, are illustrated by the following non-limiting examples.

### EXAMPLE 1

Endpoint Solubility Measurement as a Function of pH

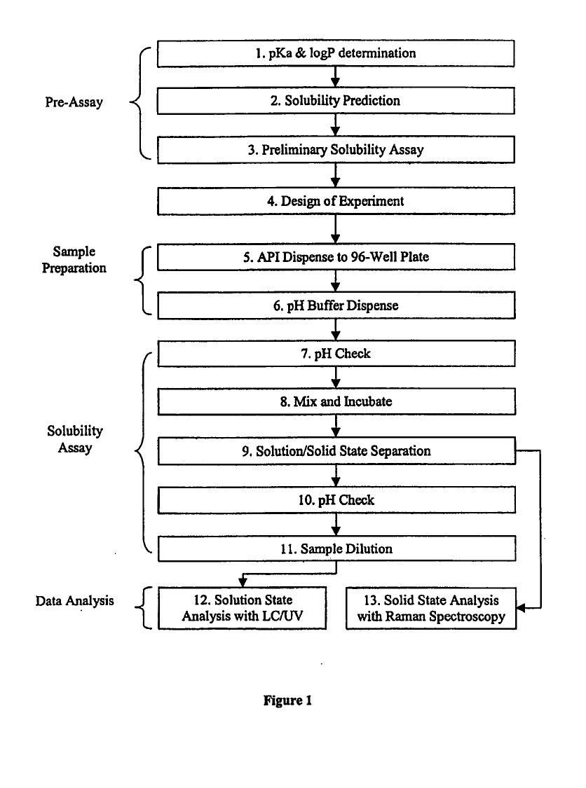

This example can be broken into steps, as shown in FIG. 1. In particular, this example was designed to determine the solubility of compounds 1 free form and 1 salt as a function of pH. Ten pH buffers were used at 5 pH values (1, 3, 5, 7, and 9) and 2 buffer strengths (50 and 200 mM). Preliminary data, obtained from step 3 of FIG. 1, was used to minimize the amount of compounds 1 free from and 1 salt used in this assay. As shown in step 5 of FIG. 1, an array of samples was prepared by dispensing controlled amounts of solid-state compounds 1 free form and 1 salt into individual wells of a polypropylene 96-well plate (Cat No. AB-0796; Abgene, Rochester N.Y.). The dispense was performed using the Autodose instrument (Autodose S A, Geneva, Switzerland). The pH condition to be tested dictated the specified dispense amounts (e.g., dispense ranged from 4.0 mg at pH 9 to 0.3 mg at pH 1).

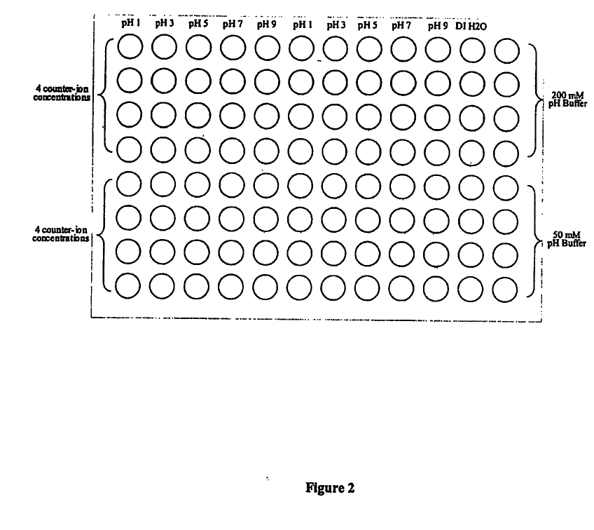

The assay was conducted by dispensing 150 microliters per well of the ten pre-made buffers at specified time points using the Tecan Genesis instrument (Tecan U.S. Inc, Research Triangle Park, N.C.). Buffers were dispensed as shown in FIG. 2, with the exception that counter-ion concentration was not tested, but a salt form was. Stir discs (Cat No. VP722F-2; V&P Scientific, Inc., San Diego, Calif.) were added to facilitate mixing and the plate was heat sealed to prevent buffer evaporation. The samples were incubated at room temperature for 3 days. After incubation, the samples were transferred to a 0.45 micrometer PVDF pore size 96 well filter plate (Cat No. 7700-1306; Whatman Inc., Clifton, N.J.) and centrifuged for 5 minutes at 3200×g to separate the solution and solid states in each well. The solution state was collected into a 96 well polypropylene receiver plate. Determination of the final pH was performed for select wells in the assay. In step 11 of FIG. 1, the samples were diluted in pH buffer into a separate 96 well polypropylene plate. A dilution factor of 4 was used. Two plates, one undiluted and one diluted, were heat sealed to prevent evaporation of the solutions. Solubility was determined by analyzing the solution state using liquid chromatography with UV detection.

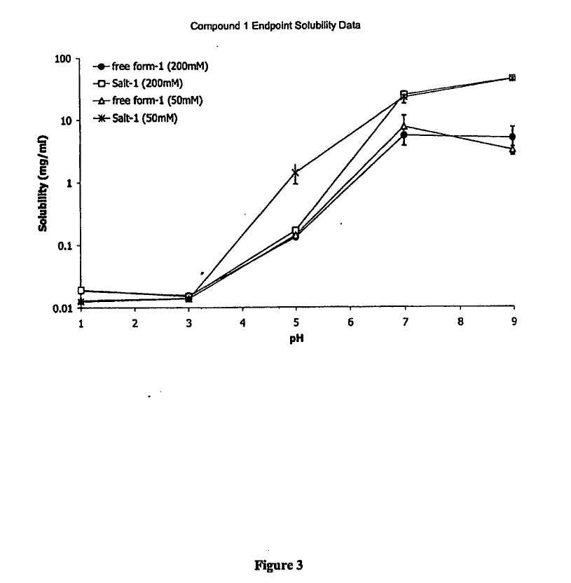

Solubility data for this assay is shown in FIG. 3. The solubility of compounds 1 free form and 1 salt increased significantly with increasing pH. The solubility of both the free form and salt leveled off in the pH 9 buffers (50 and 200 mM buffer strengths). A final pH measurement of the pH 9 buffers showed that the pH decreased from an initial value of 9 to a final value of 7. This suggested that the high solubility of the compounds at pH 9 overcame the buffer capacity of the solutions and effectively decreased the pH. This decrease in pH is responsible for the observed plateau in solubility.

### EXAMPLE 2

Solubility Determination

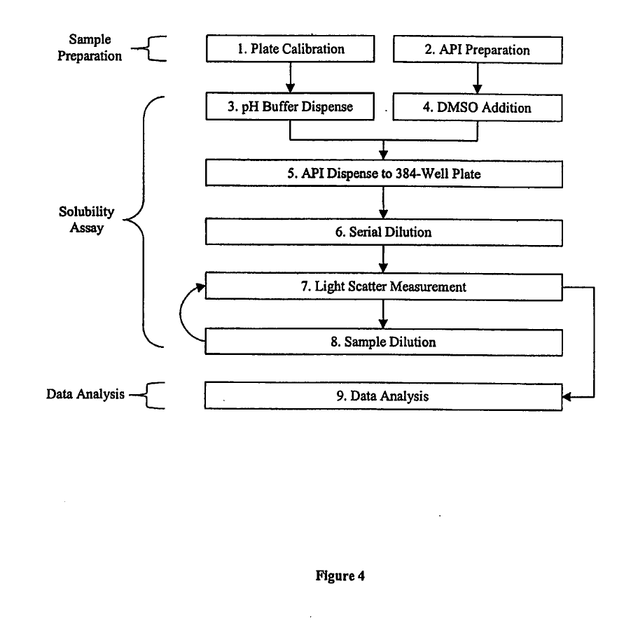

Another specific method of the invention is outlined in FIG. 4. This method utilized turbidity measurements to determine solubility and is particularly useful when the compound-of-interest is available in limited amounts.

In this example, an experiment was designed to measure the solubility of various forms of compound A in water. In the specific method shown in FIG. 4, an optically clear 384-well plate was used. In step 1 of FIG. 4, a light scatter measurement was conducted using a plate that contained 50 microliters of de-ionized (DI) water per well. This step was performed on the day prior to the assay to identify wells that contained defects and to assess the variability in light scatter for each well. The plate was then dried overnight in preparation for the assay.

Referring to step 2 of FIG. 4, samples containing various forms of compound A were prepared in DSC trays. This was done by pre-weighing the plates using a Mettler Toledo UMX2® microbalance (Mettler Toledo, Greifensee, Switzerland), and dispensing at least 50 micrograms of each different form of compound A into separate DSC trays using small spatulas (Microtools; Hampton Research, Laguna Niguel, Calif.). The trays were weighed again after the dispense step to determine the amount of compound A transferred.

The assay was started by dispensing pH buffer (50 microliters of pH 1.5 and pH 6.5 buffer) into the first column of wells in the assay plate using the Tecan® instrument described herein. In step 4 of FIG. 4, the various forms of compound A that had been deposited into the DSC trays were solubilized in 1 microliter of DMSO. Each dissolved sample was then transferred in 0.5 microliter aliquots to wells having both pH 1.5 and pH 6.5 buffer. As shown in step 6 of FIG. 4, serial dilutions of each sample were performed using the Tecan® Genesis instrument to (i) obtain a range of compound concentrations and (ii) further dilute the initial 1 percent (v/v) DMSO concentration. Eleven serial dilutions having a 30 microliter total volume were made for each initial compound A solution. Light scatter measurements of original and diluted wells were made using a NepheloStar® nephelometer (BMG Technologies, Durham, N.C.). Referring to steps 7 and 8 of FIG. 4, further dilutions of each well were performed using the Tecan® Genesis instrument and analyzed for light scatter with the nephelometer. The further dilutions served to increase the resolution in the initial concentration range of the assay. In total, four additional dilutions (i.e., step 8 of FIG. 4) were performed by adding 5, 10, 15, and 20 microliters of pH buffer in each dilution step. In this particular assay, eight different forms of compound A were assayed at two pH conditions in 30 minutes. At the end of the assay, the empty DSC plates were dried and reweighed to verify that compound A was completely transferred to the 384-well plate.

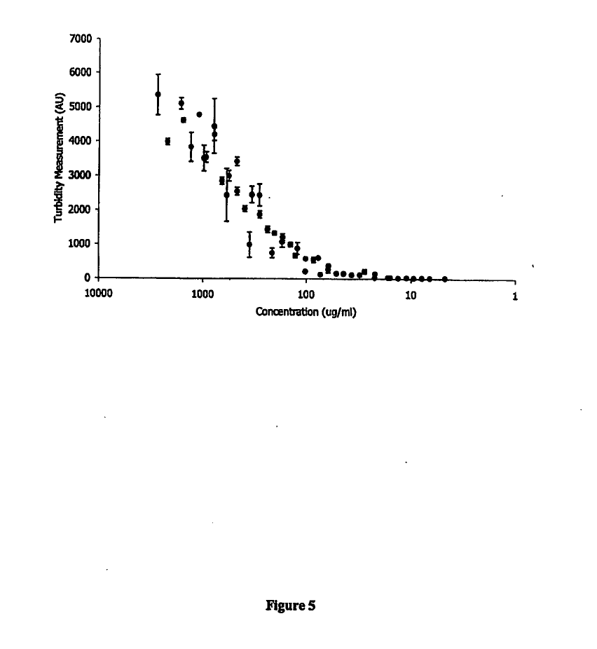

Light scatter measurements were processed in Microsoft Excel® to determine final solubility values. An example of a light scatter measurement versus concentration profile for a specific form of compound A is shown in FIG. 5. From this figure, the solubility was determined at the point where the turbidity plateaus at a minimum concentration value of compound A. As shown, the solubility of compound A was determined to be 22 micrograms/mL.

### EXAMPLE 3

Dissolution of a Compound as a Function of Counter-Ion Concentration

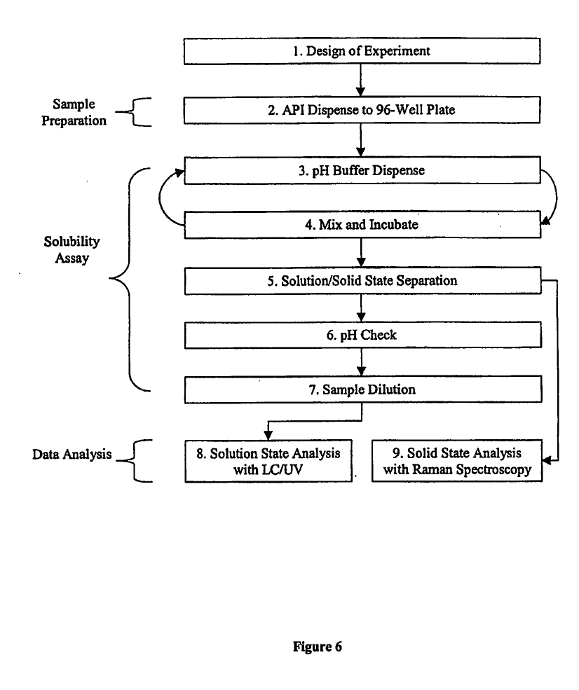

Another specific method of the invention can be divided into steps, as shown in FIG. 6. This experiment was designed for the dissolution of salt B as a function of counter-ion concentration in 0.1 N HCl buffer (i.e., pH 1). Four counter-ion conditions were to be tested in this assay: 0, 1, 3, and 10 molar equivalents. Preliminary data obtained from the endpoint solubility assay was used to minimize the amount of salt B consumed in this assay. Referring to step 2 of FIG. 6, an array of samples was prepared by dispensing 95 micrograms salt B per well into a polypropylene 96-well plate (Cat No. AB-0796; Abgene, Rochester N.Y.) using pelleting technology.

The assay was started by dispensing 150 microliters per well of the pre-made buffers (i.e., 0.1N HCl with either 0, 1, 3, or 10 molar equivalent of counter-ion) at specified time points using the Tecan® Genesis instrument (Tecan U.S. Inc, Research Triangle Park, N.C.). Buffer was dispensed at the time points one column at a time on the plate, with the longest time point dispensed first. Stir discs (Cat No. VP722F-2; V&P Scientific, Inc., San Diego, Calif.) were added following each buffer dispense to facilitate mixing and samples were sealed with removable cap strips (Cat No. AB-0981; Abgene) to prevent buffer evaporation. All samples were incubated at room temperature throughout the assay. Referring to steps 3 and 4 of FIG. 6, buffer was dispensed to the next column of wells having the next longest time point, followed by the addition of stir discs, and sealing of wells. This process was repeated until the completion of the shortest time point in the dissolution assay. The solution and solid states of all wells were then separated in step 5 by transferring the samples to a 0.45 micrometer pore size 96 well filter plate (Cat No. PN S5030; Pall Life Sciences, East Hills, N.Y.) and centrifuging for 5 minutes at 3200×g. The solution state was collected into a 96 well polypropylene receiver plate. Determination of the final pH of the solution state (step 8 of FIG. 6) was not performed in this assay. In step 7 of FIG. 6, the samples were serially diluted with methanol into separate 96 well polypropylene plates using dilution factors of 10 and 100. The plates were heat sealed to prevent evaporation of the solutions. In total, three plates were sealed for this assay, including the original plate and two dilutions. Solubility was determined by analyzing the solution state using liquid chromatography with UV detection.

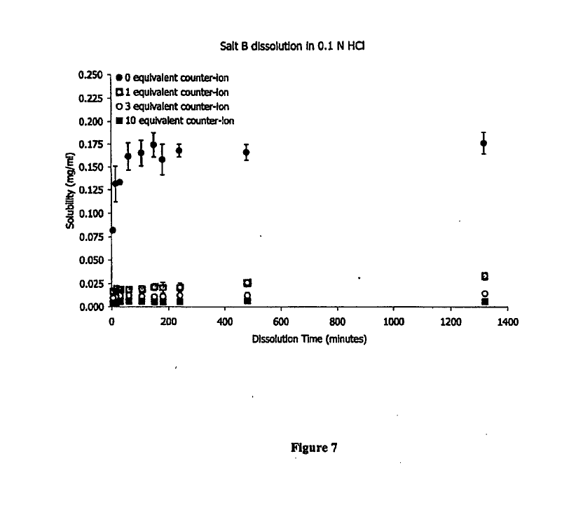

Solubility data for this assay is shown in FIG. 7. The effect of counter-ion concentration on solubility was significant. Between zero and ten equivalents of counter-ion concentration a 100-fold decrease in solubility was observed.

### EXAMPLE 4

Dissolution Assay

This example can also be characterized as having the steps shown in FIG. 6. In this case, the experiment was designed for the dissolution study of salt C. Three conditions were to be tested in this assay: dissolution of (i) C free form in 0.01 N HCl buffer (i.e., pH 2); (ii) C salt form in 0.01 N HCl buffer; and (iii) salt C with one equivalent of stabilizing agent in 0.01 N HCl buffer. Preliminary data obtained from the endpoint solubility assay was used to minimize the amount of C free form and C salt used. Based on the preliminary data, the maximum obtainable solubility was set at approximately 30 mg/ml for all three compounds of interest. The array of samples was prepared by dispensing approximately 3 mg of C (i) free form; (ii) salt; and (iii) salt with one equivalent stabilizing agent into separate wells of a 0.2 micrometer pore size, 96-well plate filter (Cat No. PN 5045, Pall Life Sciences, East Hills, N.Y.) using the Autodose instrument (Autodose S A, Geneva, Switzerland). The main advantages of performing the assay directly in the filter are that (1) the filter plate is primed and losses associated with filter plate binding of compounds on filter material is minimized; and (2) a processing step is eliminated (i.e., transfer of contents from polypropylene plate to filter plate) and the assay is performed more quickly.

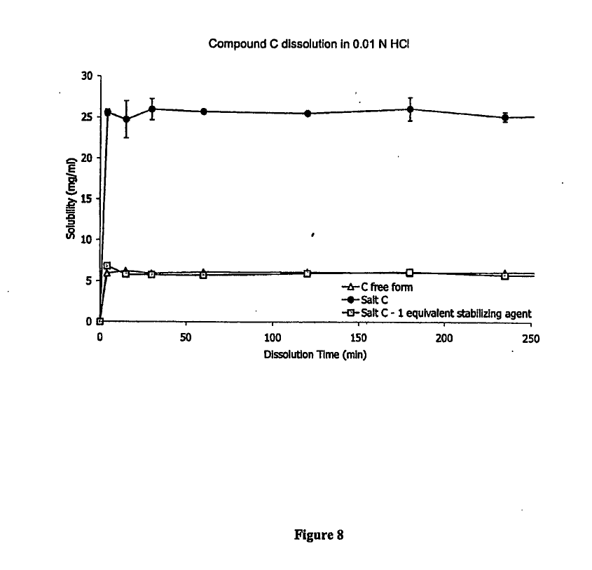

The assay was conducted by dispensing 100 microliters per well of 0.01 N HCl buffer at specified time points (FIG. 8) using the Tecan® Genesis instrument (Tecan U.S. Inc, Research Triangle Park, N.C.). Buffer was dispensed at specified time points one column at a time, with the longest time point dispensed first. Stir discs (Cat No. VP722F-2; V&P Scientific, Inc., San Diego, Calif.) were immediately added following each buffer dispense to facilitate mixing and samples were sealed to prevent buffer evaporation. Samples were incubated at room temperature throughout the assay. Referring to steps 3 and 4 of FIG. 6, buffer was then dispensed to the next column of wells having the next longest time point, followed by the addition of stir discs, and sealing of wells. This process was repeated until the completion of the shortest time point in the dissolution assay. The solution and solid states of all wells were then separated in step 5 of FIG. 6 by centrifuging the filter plate for 5 minutes at 3200×g. The solution state was collected into a 96 well polypropylene receiver plate. Final pH of the solution state was measured using a pH meter. Referring to step 7 of FIG. 6, the samples were serially diluted with a 50 percent methanol—50 percent DI water mixture (v/v) into separate 96 well polypropylene plates using dilution factors of 10 and 100. The plates were heat sealed to prevent evaporation of the solutions. In total, three plates were sealed for this assay, including the original plate and 2 dilutions. Dissolution was determined by analyzing the solution state using liquid chromatography with UV detection.

Dissolution data for this assay is shown in FIG. 8. All three forms of C obtained maximum solubility in less than 5 minutes. The lowest solubility was obtained for C free form. Salt C, which obtained the highest solubility values, had its maximum solubility limited by amount of compound dispensed into the assay plate. One equivalent of stabilizing agent negates the increased solubility of salt C.

### EXAMPLE 5

Liquid- and Solid-State Stability Measurements

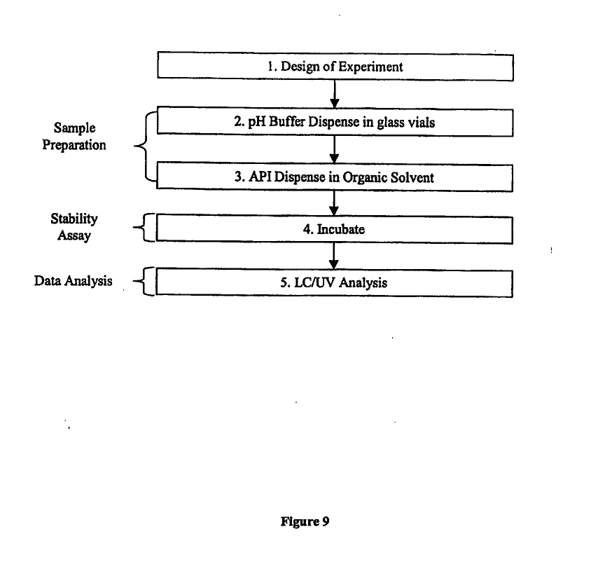

Using methods and equipment such as that described above, the solution-state stability of a compound can be determined in a method having the steps shown in FIG. 9. In this particular method, glass vials are used to contain samples of the compound and one or more organic solvents. After incubation for a set time at a set temperature (e.g., 1, 6, 12, 24, 48, or 72 hours at 25 degrees C., 40 degrees C., 50 degrees C., or 60 degrees C.), the contents of each vial are analyzed by, for example, HPLC. The presence of any degradant can be determined for each sample at some fixed time, or can be determined as a function of time, to quantify degradation.

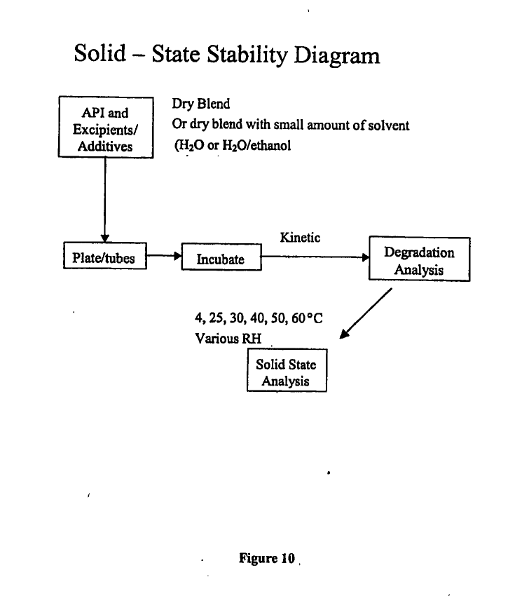

Similarly, the solid-state stability of a compound can be determined by the method shown in FIG. 10. In this case, the compound is incubated by itself or may be combined with one or more excipients prior to incubation. After incubation for a set time, set temperature, and set condition, the samples are analyzed by, for example, Raman, DSC, TGA, and HPLC to determine form changes and quantify degradants. The presence of any degradant can be determined for each sample at some fixed time, or can be determined as a function of time.

While the invention has been described with respect to the particular embodiments, it will be apparent to those skilled in the art that various changes and modifications may be made without departing from the spirit and scope of the invention as recited by the appended claims.

1. A method of determining how the solubility of a solid compound-of-interest is affected by its form which comprises:
(a) preparing an array of samples, each comprising a controlled amount of the compound-of-interest, wherein the form of the compound-of-interest in at least two of the samples is different;
(b) forming a liquid portion of each sample by adding a solvent to each sample; and
(c) determining how much compound-of-interest dissolved in the liquid portion of each sample.

2. The method of claim 1, wherein:
(a) the method further comprises separating the liquid portion of each sample from any solid portion each sample may contain prior to the determination;
(b) the solid remaining in a sample after separation of its liquid portion is analyzed to determine whether any change of form occurred;
(c) the physical form of the compound-of-interest in one sample differs from the physical form of the compound-of-interest in another sample;
(d) the compound-of-interest in one sample is amorphous and the compound-of-interest in another sample is crystalline;
(e) the compound-of-interest in one sample is crystalline and has a first crystal structure and/or a first crystal habit and the compound-of-interest in another sample is crystalline and has a second crystal structure and/or a second crystal habit, wherein the second crystal structure differs from the first crystal structure and/or the second crystal habit differs from the first crystal habit;
(f) the chemical form of the compound-of-interest in one sample differs from the chemical form of the compound-of-interest in another sample;
(g) the compound-of-interest in one sample is a salt, solvate, or co-crystal of a compound and the compound-of-interest in another sample is a different salt, solvate, or co-crystal of the compound;
(h) the compound-of-interest in one sample is a compound and the compound-of-interest in another sample is a salt, solvate, or co-crystal of the compound;
(i) the amount of compound-of-interest is less than about 100 micrograms;
(j) the amount of compound-of-interest is less than about 50 micrograms; or
(k) the amount of compound-of-interest is less than about 10 micrograms.

3. A method of determining how the dissolution of a solid compound-of-interest is affected by its form, which comprises:
(a) preparing an array of samples, each comprising a controlled amount of the compound-of-interest, wherein the form of the compound-of-interest in at least two of the samples is different;
(b) forming a liquid portion of each sample by adding a solvent to each sample; and
(c) determining how much compound-of-interest dissolved in the liquid portion of each sample as a function of time.

4. The method of claim 3, wherein:
(a) the method further comprises separating the liquid portion of each sample from any solid portion each sample may contain prior to the determination;
(b) the solid remaining in a sample after separation of its liquid portion is analyzed to determine whether any change of form occurred; of
(c) the method further comprises:
(i) preparing a first sub-array of samples, each comprising a controlled amount of the compound-of-interest in a first form;
(ii) preparing a second sub-array of samples, each comprising a controlled amount of the compound-of-interest in a second form that differs from the first form;
(iii) forming a liquid portion of each sample in the first sub-array by adding a controlled amount of a solvent to each sample in the first sub-array at a time point that is unique to each sample in the first sub-array;
(iv) forming a liquid portion of each sample in the second sub-array by adding a controlled amount of a solvent to each sample in the second sub-array at a time point that is unique to each sample in the second sub-array but is the same as the time point at which solvent was added to a sample in the first sub-array;
(v) separating the liquid portion of each sample in the first and second sub-arrays from any solid portion each sample may contain at a time point that is the same for each sample in the first and second sub-arrays; and
(vi) determining how much compound-of-interest dissolved in the liquid portion of each sample;
(d) the physical form of the compound-of-interest in one sample differs from the physical form of the compound-of-interest in another sample;
(e) the compound-of-interest in one sample is amorphous and the compound-of-interest in another sample is crystalline;
(f) the compound-of-interest in one sample is crystalline and has a first crystal structure and/or a first crystal habit and the compound-of-interest in another sample is crystalline and has a second crystal structure and/or a second crystal habit, wherein the second crystal structure differs from the first crystal structure and/or the second crystal habit differs from the first crystal habit;
(g) the chemical form of the compound-of-interest in one sample differs from the chemical form of the compound-of-interest in another sample;
(h) the compound-of-interest in one sample is a salt, solvate, or co-crystal of a compound and the compound-of-interest in another sample is a different salt, solvate, or co-crystal of the compound;
(i) the compound-of-interest in one sample is a compound and the compound-of-interest in another sample is a salt, solvate, or co-crystal of the compound;
(j) the amount of compound-of-interest is less than about 100 micrograms;
(k) the amount of compound-of-interest is less than about 50 micrograms; or
(l) the amount of compound-of-interest is less than about 10 micrograms.

5. A method of determining how the stability of a solid compound-of-interest is affected by its form, which comprises:
(a) preparing an array of samples, each comprising a controlled amount of the compound-of-interest, wherein the form of the compound-of-interest in at least two of the samples is different;
(b) exposing the compound-of-interest in each sample to a condition that may affect the stability of the compound-of-interest; and
(c) determining whether the form or chemical composition of the compound-of-interest in each sample changed.

6. The method of claim 5, wherein:
(a) the condition is pH, ionic strength, counter-ion concentration, relative humidity, radiation, oxidative conditions, mechanical stress, temperature, or time;
(b) the physical form of the compound-of-interest in one sample differs from the physical form of the compound-of-interest in another sample;
(c) the compound-of-interest in one sample is amorphous and the compound-of-interest in another sample is crystalline;
(d) the compound-of-interest in one sample is crystalline and has a first crystal structure and/or a first crystal habit and the compound-of-interest in another sample is crystalline and has a second crystal structure and/or a second crystal habit, wherein the second crystal structure differs from the first crystal structure and/or the second crystal habit differs from the first crystal habit;
(e) the chemical form of the compound-of-interest in one sample differs from the chemical form of the compound-of-interest in another sample;
(f) the compound-of-interest in one sample is a salt, solvate, or co-crystal of a compound and the compound-of-interest in another sample is a different salt, solvate, or co-crystal of the compound;
(g) the compound-of-interest in one sample is a compound and the compound-of-interest in another sample is a salt, solvate, or co-crystal of the compound;
(h) the amount of compound-of-interest is less than about 100 micrograms;
(i) the amount of compound-of-interest is less than about 50 micrograms; or
(j) the amount of compound-of-interest is less than about 10 micrograms.

7-23. (canceled)
